(19) United States
(12) Patent Application Publication
(10) Pub. No.: US 2010/0133114 A1
(43) Pub. Date: Jun. 3, 2010

(54) BIOFILM DETERRENCE IN WATER SUPPLY SYSTEMS

(75) Inventors: Shmuel Bukshpan, Ramat Ha-Sharon (IL); Gleb Zilberstein, Rechovot (IL)

Correspondence Address: The Law Office of Michael E. Kondoudis, 888 16th Street, N.W., Suite 800, Washington, DC, 20006, US

(73) Assignee: OPLON B.V., Delft (NL)

(21) Appl. No.: 12/598,431

(22) PCT Filed: Apr. 3, 2008
(86) PCT No.: PCT/IL08/00466

§ 371 Date: Nov. 19, 2009

Related U.S. Application Data
(60) Provisional application No. 60/924,152, filed on May 1, 2007.

(51) Int. Cl. C02F 1/461; A61L 2/16

(52) U.S. Cl. 205/745; 210/205

(57) ABSTRACT

Means and methods for deterring biofilm in water supply systems, comprising at least one insoluble proton sink or source (PSS). The means and methods for deterring biofilm is provided useful for killing living target cells (LTCs), or otherwise disrupting vital intracellular processes and/or intercellular interactions of the LTC upon contact. The PSS comprises, inter alia, (i) proton source or sink providing a buffering capacity; and (ii) means providing proton conductivity and/or electrical potential. The PSS is effectively disrupting the pH homeostasis and/or electrical balance within the confined volume of the LTC and/or disrupting vital intercellular interactions of the LTCs while efficiently preserving the pH of the LTCs' environment.

### FIELD OF THE INVENTION

The present invention pertains to biofilm deterrence in water supply systems. More specifically, to biofilm deterrence in water supply systems and to methods for killing living target cells, or otherwise disrupting vital intracellular processes and/or intercellular interactions of the cells, while efficiently preserving the pH of the cells environment.

### BACKGROUND OF THE INVENTION

Biofilm formation in water systems has important public health implications. Drinking water systems are known to harbor biofilms, even though these environments often contain disinfectants. Any system providing an interface between a surface and a fluid has the potential for biofilm development. Water cooling towers for air conditioners are well-known to pose public health risks from biofilm formation, as episodic outbreaks of infections like Legionnaires' disease attest. Biofilms have been identified in flow conduits like hemodialysis tubing, and in water distribution conduits. Biofilms have also been identified to cause biofouling in selected municipal water storage tanks, private wells and drip irrigation systems, unaffected by treatments with up to 200 ppm chlorine. Biofilms are a constant problem in food processing environments.

Several methods have been proposed to prevent and destroy biofilms in water supply systems, including mechanical (e.g. rasping, sonication, freezing and thawing), chemical (e.g. biocides, detergents, surfactants) and enzymatic means.

### Mechanical and Physical Processes

Recent patents have described systems associating filtration techniques to ultraviolet (UV) radiation. In the range of 200-300 nm, UV radiation is very effective in killing micro-organisms, and UV lamps have been extensively used, since almost no by-products are produced, contrarily to what happens during chlorination and ozonation. To destroy and prevent biofilms inside conduits, Korin (2004, US20046773610) presented an integrated filtration and (Aussudre, C., sterilization unit using a wavelength (120-242 nm) of the UV light source that enabled both the sterilisation of the previously filtrated liquid and the generation of ozone from an oxygen containing gas. Albelda and co-workers (1999, U.S. Pat. No. 5,925,257.) had suggested a method that included filtration and/or exposure of the liquid (to be used inside tubes) to UV radiation prior addition of an oxygen solution containing at least 0.2% (w/w) oxygen. Costerton (1983, U.S. Pat. No. 4,419,248) proposed to freeze the biofilm in fouled pipes sufficiently slow to allow the formation of large (0.5-20 mm) sharp-edged ice crystals within the polysaccharide matrix. To achieve this, the pipes may be cooled with dry ice or liquid nitrogen. The frozen biofilm is subsequently thawed and removed, for instance, by a liquid flow through the pipes.

The opposite situation, the use of high temperatures, may also be used to disinfect surfaces and prevent the formation of biofilms. Micro-organisms such as Legionella can be eradicated from domestic hot water supply systems and similar devices, connected to water distribution circuits, using a heating cell that may also be used to heat the primary circuit (Aussudre, C., et al., 2006, WO06037868A1). In sea vessels and exterior places, the temperature of the pipes may be considerably lower than inside heated water tanks, decreasing the success of the process. In such cases, a heating ribbon, wire, rod or an elongated heating spiral may be applied inside the tubes to maintain the water at an efficient temperature (around 60° C.), while decreasing energy and water consumption (Korstanje, J. C., 2006; WO06059898A1). By circulating heated water at around 80° C. through the fluid circuit of a dialysis machine comprising a water treatment module, a dialysate preparation module and circuit and an extracorporeal circuit (including the dialyser, arterial and venous blood lines to connect to the patient) during a certain period (around one hour) disinfection of all circuits may be achieved (Kenley, R. S., et al., 1997, U.S. Pat. No. 5,591,344). Suddath and co-workers developed a self-cleaning system with a boiler to provide sanitized water or steam to a dental workstation [Suddath, J. N., et al., 2004, U.S. Pat. No. 6,821,480). The steam is used to sterilise the delivery line and workstation and destroy adherent cells.

Small diameter tubes are difficult to clean, especially if the tubes are long, because fouling decreases flow velocities. Haemodialysis hollow fibers have length/diameter (L/D) ratios of about 1000-1500 and tubular membranes of 500-1500, dental chair tubes have L/D of 2000-3000, industrial pipes have usually L/D ratios of 1000-3000 and in endoscopes the ratio is about 500-2000 (Tabani, Y., and Labib, M. E (2005) US20056945257). Tabani and coworkers patented a process for removing adherent contaminants from hollow porous fibres which consists in back-flushing a liquid to fill the pores and application of a gas flow. The mixture of gas and bubbles provokes enough turbulence to remove the adherent particles into the liquid phase. The process may be applied in tubes with diameters from c.a. 0.2 mm to 10 cm or more, depending on a sufficient gas supply. Bubbles are able to remove mature biofilms at the point collision due to the combined effect of fluid dynamic shear forces and thermodynamic forces that pull bacteria from a surface when the bubble contacts the biofilm (Parini M R, and Pitt W G., 2006, Colloids Surf B 52: 39-46). The fraction of biofilm removed per bubble is about 0.4 and this technique may be applied by powered toothbrushes to remove bacterial biofilms from teeth.

### Chemical Processes

The application of oxidation processes, e.g. through the usage of oxidants such as ozone, hydrogen peroxide, chlorine or chlorine dioxide, is a well known process of water treatment, being able to remove organic and inorganic compounds in water while improving taste and colour.

However, ozone low water solubility and stability, high cost and inefficiency to oxidise some organic compounds hamper its application, in particular economically (Kasprzyk-Hordem B, et al., (2003) Appl Catal B Environ 46: 639-669). Electrochemical generation of ozone for “point-of-use” applications (osmosis systems, refrigerators, drinking fountains, etc) to provide disinfected water, ozonecontaining water and/or ozone gas was presented by Andrews and co-workers (Andrews, C. C., et al., 2002, US20026458257). Disinfected water, produced by introduction of ozone into purified water, finds its application in anti-microbial and cleansing applications at consumer-level, e.g., to wash food, cloths, toys, bathrooms, etc, as well as to wash and disinfect medical devices. Water enriched in ozone is also effective in eliminating microorganisms and prevent biofilm formation in water circuits delivering water to a patient's mouth during dental procedures (Engelhard, R. and Kasten, S. P., 1999, U.S. Pat. No. 5,942,125). In ozone treatment of dialysis feed-water, the ozone should be applied to the water storage tank and removed prior the use of the water in dialysis treatment using UV light (Van Newenhizen, J., 1996, U.S. Pat. No. 5,585,003). Water for dialysis and other processes requiring ultra-pure water can also be cleaned and disinfected by maintaining an acidic pH with a high carbon dioxide concentration in solution (Smith, S. D., 2005, US20056908546). Chlorine dioxide is a gas with effective disinfectant, bleaching and oxidizing properties, although explosive in contact with air at concentrations above 10%. Kross and coworkers suggested the application of 25-2500 ppm chlorine dioxide solutions to decontaminate small diameter water pipes (such as those in dental units, ranging 6-19 mm) and concentrations 1-10 ppm to maintain the circuit clean (Kross, R. D., and Wade, W., 2003, US20036599432).

To control biofilms and micro-organisms in general in systems supplying water to large medical or dental devices, a filtration system containing filters to remove particles, organic matter and bacteria can be combined with a pressurised storage tank to which antimicrobial agents are applied (Chandler, J. W., 2002, US20026423219). The biocidal agent being a mixture of hydroperoxide ions, a phase transfer catalyst and a trace colour or an antiseptic agent from citrus fruits, such as grapefruit seed extract.

### Enzymatic Action

The polymeric matrix that anchors the cells constitutes a penetration barrier to biocides, decreasing their potency in comparison to that observed with planktonic cells while promoting microbial resistance (Marion-Ferey K, et al., (2003) J Hosp Infect 53: 64-71). The cells inside the biofilm have a lower access to nutrients and thus a slower growth rate, becoming more protected to the majority of antibiotics and biocidal agents since they act primarily upon dividing cells. The use of substances capable of destroying the physical integrity of the matrix, interfere with bacterial adhesion or initiate cell detachment from surfaces are good alternatives to biocides and/or disinfectants. The latter contribute to the propagation and spread of resistant strains (Ofek I, et al., (2003) FEMS Immunol Med Microbiol 38: 181-91) and its use may be restricted by environmental regulations (Chen X, and Stewart P S., (2000) Water Res 34: 4229-33). Especial attention has been given to enzymes able to destroy polysaccharides, which are the primary building blocks of slime. Among these are proteases, such as alkaline proteases, and a-amylases from various Bacillus strains Gupta R, et al., (2002) Appl Microbiol Biotechnol 59: 13-32) acidic proteases and glucoamylases from Aspergillus niger (Orgaz B, et al., Enz Microb Technol (in press)) and acidic and alkaline proteases from pineapple stem and cellulases (Napper A D, et al., (1994) Biochem J 301: 727-35). In the late 1980s and early 1990s, combinations of biocides and enzymes were used: the enzymes were responsible for destroying the polysaccharide matrix to enhance the biocide action. Pedersen and Hatcher (1987, U.S. Pat. No. 4,684,469), used methylene-bis-thiocyanate, dimethyl dithiocarbamare or disodium ethylene-bis-dithiocarbamate as biocide and amylase, a dextran degrading enzyme or a levan hydrolase as the polysaccharide degrading enzyme. Robertson et al. (1994), (U.S. Pat. No. 5,324,432) proposed the application of chlorine, hypochlorite, bromine, hydrogen peroxide, etc., in a concentration of 0.5-500 ppm and trypsin and/or endo-protease and/or chymotrypsin in about 0.01-1000 units to inhibit the growth of filamentous organisms. More environment friendly solutions have been proposed since then, including: (i) mixtures of enzymes and a surface active agent, preferentially anionic (Hollis, C. G., et al., 1995, U.S. Pat. No. 5,411,666); (ii) at least one enzyme belonging to carbohydrases, proteases, glycol proteases or lipases and a short-chained glycol component [Eyers, M. E., et al., (1998) U.S. Pat. No. 5,789,239); (iii) enzyme blending in 2-100 ppm of cellulase, a-amylase and protease (Wiatr, C. L., (1990), U.S. Pat. No. 4,936,994).

The increasing understanding of how a biofilm is formed and the role of each mechanism involved in cell adhesion is providing precious information to the development of sound strategies to combat cell colonisation. Interferences (i) in the initially cell-to-surface and cell-to-cell contact, responsible for the formation of the first microcolonies at the surface, (ii) with the molecules responsible for cell-to-cell communication or quorum sensing and (iii) with the formation of EPS, responsible for the structure of the biofilm, can disrupt the process of biofilm formation and proliferation.

The approach and adhesion of cells to surfaces is facilitated by the cell surface hydrophobicity (van Loosdrecht M C M, et al., (1987) Appl Environ Microbiol 53: 1893-97.; Yaskovich G A., (1998) Appl Biochem Microbiol 34: 373-76 and Kos B, et al., (2003) J Appl Microbiol 94: 981-87). Cells growing in alcohols, hydrocarbons and terpenes, as sole carbon and energy sources, are able to change their surface hydrophobicity (de Carvalho C C C R, et al., (Appl Microbiol Biotechnol 67: 383-88 2005). The routinely use of biocides in industrial and household products can act as a selective pressure upon exposed bacteria. The more tolerant individuals will, in that case, have an increased contribution to the reproduction of the population under stress (Mulvey M, and Diamond S A. (1991) In: Newman M C, McIntosh A W Eds, Metal Ecotoxicology. Chelsea, Lewis. 301-21). This could be responsible to the observed increased number of hospital infections (White D G, and McDermott P F., (2001) Curr Opin Microbiol 4: 313-17). Development of biocides and their extensive application should be conscientious and aimed at health and environment friendly, effective and economically feasible compositions.

The above described studies and patents are aimed mainly for the removal of existing and well established biofilms in water systems. However, a more efficient and economically worthwhile approach would be to prevent biofilm formation in the first place. To be able to achieve this, there exists a need to be able to render general surfaces bactericidal. There is a keen interest in materials capable of killing harmful microorganisms upon contact thereby, preventing the very first step in the cascade of biofilm formation, namely bacterial adhesion to the surface and establishment, from occurring. Since ordinary materials are not antimicrobial or cell-killing as such, their modification is required. For example, surfaces chemically modified with poly(ethylene glycol) and certain other synthetic polymers can repel (although not kill) microorganisms (Bridgett, M. J., et al., (1992) Biomaterials 13, 411-416; Arciola, C. R., et al Alvergna, P., Cenni, E. & Pizzoferrato, A. (1993) Biomaterials 14, 1161-1164; Park, K. D., Kim, Y. S., Han, D. K., Kim, Y. H., Lee, E. H. B., Suh, H. & Choi, K. S. (1998) Biomaterials 19, 51-859.). Alternatively, materials can be impregnated with antimicrobial agents, such as antibiotics, quarternary ammonium compounds, silver ions, or iodine, that are gradually released into the surrounding solution over time and kill deleterious cells and microorganisms there (Medlin, J. (1997) Environ. Health Preps. 105, 290-292; Nohr, R. S. & Macdonald, G. J. (1994) J. Biomater. Sci., Polymer Edn. 5, 607-619 Shearer, A. E. H., et al (2000) Biotechnol. Bioeng 67, 141-146.). There exist polymers with inherent antimicrobial or antistatic properties. Such polymers can be applied or used in conjunction with a wide variety of substrates (e.g., glass, textiles, metal, cellulosic materials, plastics, etc.) to provide the substrate with antimicrobial and/or antistatic properties. In addition, the polymers can also be combined with other polymers to provide such other polymers with antimicrobial and/or antistatic properties.

However, there is also a need for such agents to be both sustainable and to be compatible, and to be used on and with a wide variety of polymer materials and substrates. Various additives and polymer systems have been suggested as providing antimicrobial properties. See, for example, U.S. Pat. No. 3,872,128 to Byck, U.S. Pat. No. 5,024,840 to Blakely et al, U.S. Pat. No. 5,290,894 to Malrose et al, U.S. Pat. Nos. 5,967,714, 6,203,856 and U.S. Pat. No. 6,248,811 to Ottersbach et al, U.S. Pat. No. 6,194,530 to Klasse et al. and U.S. Pat. No. 6,242,526 to Siddiqui et al. There, however, remains a need for potentially less toxic polymer compositions that provide sustainable cell killing properties to a wide variety of substrates and materials.

It is quite well known that charged molecules in solution are able to kill bacteria (Endo et al., 1987; Fidai et al., 1997; Friedrich et al., 2000; Isquith et al., 1972). However, it has been realized more recently that charges attached to surfaces can kill bacteria upon contact. All bear cationic, positively charged groups, such as quaternary ammonium (Thome et al., 2003) or phosphonium (Kanazawa et al., 1993; Popa et al., 2003). Various architectures have been tested: self-assembled monolayers (Atkins, 1990; Gottenbos et al., 2002; Rondelez & Bezou, 1999), polyelectrolyte layers (Lee et al., 2004; Lin et al., 2002, 2003; Popa et al., 2003; Sauvet et al., 2000; Thome et al., 2003; Tiller et al., 2001) and hyperbranched dendrimers (Cen et al., 2003; Chen & Cooper, 2000, 2002).

The following publications are incorporated as a reference for the present invention, namely Albelda, D., Moshe, K.: U.S. Pat. No. 5,925,257 (1999); Andrews, C. C., Murphy, O. J., Hitchens, G. D.: US20026458257 (2002); Arciola, C. R., Alvergna, P., Cenni, E. & Pizzoferrato, A. (1993) Biomaterials 14, 1161-1164; Atkins, P. W. (1990). Physical Chemistry. New York: W. H. Freeman & Company; Aussudre, C., Berthou, M., Chopard, F.: WO06037868A1 (2006); Boring et al., CA Cancer Journal for Clinicians. 43:7 1993; Bridgett, M. J., et al., (1992). Biomaterials 13, 411-416; Cen, L., Neoh, K. G. & Kang, E. T. (2003). Langmuir 19, 10295-10303; Chandler, J. W.: US20026423219 (2002);

Chen, C. Z. & Cooper, S. L. (2000). Adv Materials 12, 843-846; Chen, C. Z. & Cooper, S. L. (2002). Biomaterials 23, 3359-3368; Chen X, Stewart P S. Biofilm removal caused by chemical treatments. Water Res 2000; 34: 4229-33; Costerton, J. W. F.: U.S. Pat. No. 4,419,248 (1983); De Carvalho C C C R, Parreño-Marchante B, Neumann G, da Fonseca M M R, Heipieper H J. Adaptation of Rhodococcus erythropolis DCL14 to growth on n-alkanes, alcohols and terpenes. Appl Microbiol Biotechnol 2005; 67: 383-88; Endo, Y., Tani, T. & Kodama, M. (1987). Appl Environ Microbiol 53, 2050-2055; Engelhard, R., Kasten, S. P.: U.S. Pat. No. 5,942,125 (1999); Eyers, M. E., Van Pee, K. L. I., Van Poele, J., Schuetz, J. F., Schenker, A. P.: U.S. Pat. No. 5,789,239 (1998); Fidai, S., Farer, S. W. & Hancock, R. E. (1997). Methods Mol Biol 78, 187-204; Friedrich, C. L., Moyles, D., Beverige, T. J. & Hancock, R. E. W. (2000). Antimicrob Agents Chemother 44, 2086-2092; Gottenbos, B., van der Mei, H. C., Klafter, F., Nieuwenhuis, P. & Busscher, H. J. (2002). Biomaterials 23, 1417-1423; Gupta R, Beg Q K, Lorenz P. Bacterial alkaline proteases: molecular approaches and industrial applications. Appl Microbiol Biotechnol 2002; 59: 13-32.; Hollis, C. G., Terry, J. P., Jaquess, P. A.: U.S. Pat. No. 5,411,666 (1995); Isquith, A. J., Abbott, E. A. & Walters, P. A. (1972). Appl Microbiol 24, 859-863; Kanazawa, A., Ikeda, T. & Endo, T. (1993). J Polym Sci Part A Polym Chem 31, 1467-1472; Kasprzyk-Hordern B, Ziólek M, Nawrocki J. Catalytic ozonation and methods of enhancing molecular ozone reactions in water treatment. Appl Catal B Environ 2003; 46: 639-669; Kenley, R. S., Treu, D. M., Peter, Jr., F. H., Feldsein, T. M., Pawlak, K. E., Adolf, W. F., Roettger, L.: U.S. Pat. No. 5,591,344 (1997); Korin, A.: US20046773610 (2004); Korstanje, J. C.; WO06059898A1 (2006); Kos B, Suskovic J, Vukovic S, Simpraga M, Frece J, Matosic S. J Appl Microbiol 2003; 94: 981-87; Kross, R. D., Wade, W.: US20036599432 (2003); Lee, S. B., Koepsel, R. R., Morley, S. W., Matyajaszewski, K., Sun, Y. & Russell, A. J. (2004). Biomacromolecules 5, 877-882; Lin, J., Qiu, S., Lewis, K. & Klibanov, A. M. (2002). Biotechnol Prog 18, 1082-1096; Lin, J., Qiu, S., Lewis, K. & Klibanov, A. M. (2003). Biotechnol Bioeng 83, 168-172; Medlin J. 1997. Germ warfare. Environ Health Persp 105:290-292.; Marion-Ferey K, Pasmorey M, Stoodleyy P, Wilsony S, Husson G P, Costerton J W. Biofilm removal from silicone tubing: an assessment of the efficacy of dialysis machine decontamination procedures using an in vitro model. J Hosp Infect 2003; 53: 64-71; Mulvey M, Diamond S A. In: Newman M C, McIntosh A W Eds, Metal Ecotoxicology. Chelsea, Lewis. 1991; 301-21; Napper A D, Bennett S P, Borowski M, Holdridge M B, Leonard M J C, Rogers E E, Duan Y, Laursen R A, Reinhold B, Shames S L. Purification and characterization of multiple forms of the pineapple-stem-derived cysteine proteinases ananain and comosain. Biochem J 1994; 301: 727-35; Nohr R S and Macdonald G J. 1994. J Biomater Sci, Polymer Edn 5:607-619; Ofek I, Hasty D L, Sharon N. FEMS Immunol Med Microbiol 2003; 38: 181-91; Orgaz B, Kives J, Pedregosa A M, Monistrol I F, Laborda F, SanJosé C. Bacterial biofilm removal using fungal enzymes. Enz Microb Technol (in press); Parini M R, Pitt W G. Dynamic removal of oral biofilms by bubbles. Colloids Surf B 2006; 52: 39-46; Park, K. D., Kim, Y. S., Han, D. K., Kim, Y. H., Lee, E. H. B., Suh, H. & Choi, K. S. (1998) Biomaterials 19, 51-859; Pedersen, D. E., Hatcher, H. J.: U.S. Pat. No. 4,684,469 (1987); Popa, A., Davidescu, C. M., Trif, R., Ilia, G h., Iliescu, S. & Dehelean, G h. (2003). React Funct Polym 55, 151-158; Robertson, L. R., LaZonby, J. G., Krolczyk, J. J., Melo, H. R.: U.S. Pat. No. 5,324,432 (1994); Rondelez, F. & Bezou, P. (1999). Actual Chim 10, 4-8; Shearer, A. E. H., et al., (2000), Biotechnol. Bioeng 67, 141-146; Smith, S. D.: US20056908546 (2005); Suddath, J. N., Piskorowski, W., Kasbrick, J. J.: U.S. Pat. No. 6,821,480 (2004); Tabani, Y., Labib, M. E.: US20056945257 (2005); Thome, J., Holländer, A., Jaeger, W., Trick, I. & Oehr, C. (2003). Surface Coating Technol 174-175, 584-587; Tiller, J. C., Liao, C., Lewis, K. & Klibanov, A. M. (2001). Proc Natl Acad Sci USA 98, 5981-5985; Van Loosdrecht M C M, Lyklema J, Norde W, Schraa G, Zehnder A J B. The role of bacterial cell wall hydrophobicity in adhesion. Appl Environ Microbiol 1987; 53: 1893-97; Van Newenhizen, J.: U.S. Pat. No. 5,585,003 (1996); Wiatr, C. L.: U.S. Pat. No. 4,936,994 (1990); White D G, McDermott P F. Biocides, drug resistance and microbial evolution. Curr Opin Microbiol 2001; 4: 313-17; and, Yaskovich G A. The role of cell surface hydrophobicity in adsorption immobilization of bacterial strains Appl Biochem Microbiol 1998; 34: 373-76.

An important advantage of this approach is that the biocidal molecules are attached covalently to the substrates, which allows their reusability after cleaning processes and prevents uncontrolled material release to the environment. However, the key parameters of the effects involved in the biocidal process have not yet been identified. There thus remains a need for and it would be highly advantageous to have agents capable of sustained and long-acting antimicrobial activity both against biofilm-forming microorganisms.

### SUMMARY OF THE INVENTION

It is hence one object of the present invention to disclose a cost effective means for deterring biofilm in water supply systems, comprising at least one insoluble proton sink or source (PSS), said means for deterring biofilm is provided useful for killing living target cells (LTCs), or otherwise disrupting vital intracellular processes and/or intercellular interactions of said LTC upon contact; said PSS comprising (i) proton source or sink providing a buffering capacity; and (ii) means providing proton conductivity and/or electrical potential; wherein said PSS is effectively disrupting the pH homeostasis and/or electrical balance within the confined volume of said LTC and/or disrupting vital intercellular interactions of said LTCs while efficiently preserving the pH of said LTCs' environment.

It is in the scope of the invention wherein the PSS is an insoluble hydrophobic, either anionic, cationic or zwitterionic charged polymer, useful for killing living target cells (LTCs), or otherwise disrupting vital intracellular processes and/or intercellular interactions of the LTC upon contact. It is additionally or alternatively in the scope of the invention, wherein the PSS is an insoluble hydrophilic, anionic, cationic or zwitterionic charged polymer, combined with water-immiscible polymers useful for killing living target cells (LTCs), or otherwise disrupting vital intracellular processes and/or intercellular interactions of the LTC upon contact. It is further in the scope of the invention, wherein the PSS is an insoluble hydrophilic, either anionic, cationic or zwitterionic charged polymer, combined with water-immiscible either anionic, cationic of zwitterionic charged polymer useful for killing living target cells (LTCs), or otherwise disrupting vital intracellular processes and/or intercellular interactions of the LTC upon contact.

It is also in the scope of the invention wherein the PSS is adapted in a non-limiting manner, to contact the living target cell either in a bulk or in a surface; e.g., at the outermost boundaries of an organism or inanimate object that are capable of being contacted by the PSS of the present invention; at the inner membranes and surfaces of microorganisms, animals and plants, capable of being contacted by the PSS by any of a number of transdermal delivery routes etc; at the bulk, either a bulk provisioned with stirring or nor etc.

It is further in the scope of the invention wherein either (i) a PSS or (ii) an article of manufacture comprising the PSS also comprises an effective measure of at least one additive.

It is another object of the present invention to disclose the aforesaid means, wherein said proton conductivity is provided by water permeability and/or by wetting, especially wherein said wetting is provided by hydrophilic additives.

It is another object of the present invention to disclose the aforesaid means, wherein said proton conductivity or wetting is provided by inherently proton conductive materials (IPCMs) and/or inherently hydrophilic polymers (IHPs), selected from a group consisting of sulfonated tetrafluortheylene copolymers; sulfonated materials selected from a group consisting of silica, polythion-ether sulfone (SPTES), styrene-ethylene-butylene-styrene (S-SEBS), polyether-ether-ketone (PEEK), poly (arylene-ether-sulfone) (PSU), Polyvinylidene Fluoride (PVDF)-grafted styrene, polybenzimidazole (PBI) and polyphosphazene; proton-exchange membrane made by casting a polystyrene sulfonate (PSSnate) solution with suspended micron-sized particles of cross-linked PSSnate ion exchange resin; commercially available Nafion™ and derivatives thereof.

It is another object of the present invention to disclose the aforesaid means, wherein the means comprises two or more, either two-dimensional (2D) or three-dimensional (3D) PSSs, each of which of said PSSs consisting of materials containing highly dissociating cationic and/or anionic groups (HDCAs) spatially organized in a manner which efficiently minimizes the change of the pH of the TLC's environment; each of said HDCAs is optionally spatially organized in specific either 2D, topologically folded 2D surfaces, or 3D manner efficiently which minimizes the change of the pH of the TLC's environment; further optionally, at least a portion of said spatially organized HDCAs are either 2D or 3D positioned in a manner selected from a group consisting of (i) interlacing; (ii) overlapping; (iii) conjugating; (iv) either homogeneously or heterogeneously mixing; and (iv) tiling the same.

It is acknowledged in this respect to underline that the term HDCAs refers, according to one specific embodiment of the invention, and in a non-limiting manner, to ion-exchangers, e.g., water immiscible ionic hydrophobic materials.

It is another object of the present invention to disclose the aforesaid means wherein said PSS is effectively disrupting the pH homeostasis within a confined volume while efficiently preserving the entirety of said TLC's environment; and further wherein said environment's entirety is characterized by parameters selected from a group consisting of said environment functionality, chemistry; soluble's concentration, possibly other then proton or hydroxyl concentration; biological related parameters; ecological related parameters; physical parameters, especially particles size distribution, rehology and consistency; safety parameters, especially toxicity, otherwise LD50 or ICT50 affecting parameters; olphactory or organoleptic parameters (e.g., color, taste, smell, texture, conceptual appearance etc); or any combination of the same.

It is another object of the present invention to disclose the aforesaid means wherein the means is provided useful for disrupting vital intracellular processes and/or intercellular interactions of said LTC, while both (i) effectively preserving the pH of said TLC's environment and (ii) minimally affecting the entirety of the TLC's environment such that a leaching from said PSS of either ionized or neutral atoms, molecules or particles (AMP) to the TLC's environment is minimized.

It is well in the scope of the invention wherein the aforesaid leaching minimized such that the concentration of leached ionized or neutral atoms is less than 1 ppm. Alternatively, the aforesaid leaching is minimized such that the concentration of leached ionized or neutral atoms is less than 50 ppb. Alternatively, the aforesaid leaching is minimized such that the concentration of leached ionized or neutral atoms is less than 50 ppb and more than 10 ppb. Alternatively, the aforesaid leaching is minimized such that the concentration of leached ionized or neutral atoms is less than 10 but more than 0.5 ppb. Alternatively, the aforesaid leaching is minimized such that the concentration of leached ionized or neutral atoms is less than 0.5 ppb.

It is another object of the present invention to disclose the aforesaid means wherein the means is provided useful for disrupting vital intracellular processes and/or intercellular interactions of said LTC, while less disrupting pH homeostasis and/or electrical balance within at least one second confined volume (e.g., non-target cells or viruses, NTC).

It is another object of the present invention to disclose the aforesaid means wherein the means is provided wherein said differentiation between said TLC and NTC is obtained by one or more of the following means (i) providing differential ion capacity; (ii) providing differential pH values; and, (iii) optimizing PSS to target cell size ratio; (iv) providing a differential spatial, either 2D, topologically folded 2D surfaces, or 3D configuration of said PSS; (v) providing a critical number of PSS' particles (or applicable surface) with a defined capacity per a given volume; and (vi) providing size exclusion means.

It is another object of the present invention to disclose the aforesaid means wherein the means comprising at least one insoluble non-leaching PSS according as defined above; said PSS, located on the internal and/or external surface of said means for deterring biofilm, is provided useful, upon contact, for disrupting pH homeostasis and/or electrical balance within at least a portion of an LTC while effectively preserving pH & functionality of said surface.

It is another object of the present invention to disclose the aforesaid means wherein the means is having at least one external proton-permeable surface with a given functionality (e.g., electrical current conductivity, affinity, selectivity etc), said surface is at least partially composed of, or topically and/or underneath layered with a PSS, such that disruption of vital intracellular processes and/or intercellular interactions of said LTC is provided, while said TLC's environment's pH & said functionality is effectively preserved.

It is another object of the present invention to disclose the aforesaid means wherein the means comprising a surface with a given functionality, and one or more external proton-permeable layers, each of which of said layers is disposed on at least a portion of said surface; wherein said layer is at least partially composed of or layered with a PSS such that vital intracellular processes and/or intercellular interactions of said LTC are disrupted, while said TLC's environment's pH & said functionality is effectively preserved.

It is another object of the present invention to disclose the aforesaid means wherein the means comprising (i) at least one PSS; and (ii) one or more preventive barriers, providing said PSS with a sustained long activity; preferably wherein at least one barrier is a polymeric preventive barrier adapted to avoid heavy ion diffusion; further preferably wherein said polymer is an ionomeric barrier, and particularly a commercially available Nafion™.

It is acknowledged in this respect that the presence or incorporation of barriers that can selectively allow transport of protons and hydroxyls but not of other competing ions to and/or from the solid ion exchange (SIEx) surface eliminates or substantially reduces the ion-exchange saturation by counter-ions, resulting in sustained and long acting cell killing activity of the materials and compositions of the current invention.

It is in the scope of the invention, wherein the proton and/or hydroxyl-exchange between the cell and strong acids and/or strong basic materials and compositions may lead to disruption of the cell pH-homeostasis and consequently to cell death. The proton conductivity property, the volume buffer capacity and the bulk activity are pivotal and crucial to the present invention.

It is further in the scope of the invention, wherein the pH derived cytotoxicity can be modulated by impregnation and coating of acidic and basic ion exchange materials with polymeric and/or ionomeric barrier materials.

It is another object of the present invention to disclose the aforesaid means wherein the means is adapted to avoid development of TLC's resistance and selection over resistant mutations.

It is another object of the present invention to disclose the aforesaid means wherein the means is designed as a continuous barrier said barrier is selected from a group consisting of either 2D or 3D membranes, filters, meshes, nets, sheet-like members or a combination thereof.

It is another object of the present invention to disclose the aforesaid means wherein the means is designed as an insert, comprising at least one PSS, said insert is provided with dimensions adapted to ensure either (i) reversibly mounting or (ii) permanent accommodation of said insert within a predetermined article of manufacture.

It is another object of the present invention to disclose the aforesaid means wherein the means is characterized by at least one of the following (i) regeneratable proton source or sink; (ii) regeneratable buffering capacity; and (iii) regeneratable proton conductivity.

It is another object of the present invention to disclose a method for killing living target cells (LTCs), or otherwise disrupting vital intracellular processes and/or intercellular interactions of said LTC being in a means for deterring biofilm , especially cosmetic or foodstuffs' means for deterring biofilm; said method comprising steps of providing said means for deterring biofilm with at least one PSS having (i) proton source or sink providing a buffering capacity; and (ii) means providing proton conductivity and/or electrical potential; contacting said LTCs with said PSS; and, by means of said PSS, effectively disrupting the pH homeostasis and/or electrical balance within said LTC while efficiently preserving the pH of said LTC's environment.

It is another object of the present invention to disclose a method as defined above, wherein said step (a) further comprising a step of providing said PSS with water permeability and/or wetting characteristics, in particular wherein said proton conductivity and wetting is at least partially obtained by providing said PSS with hydrophilic additives.

It is another object of the present invention to disclose a method as defined in any of the above, wherein the method further comprising a step of providing the PSS with inherently proton conductive materials (IPCMs) and/or inherently hydrophilic polymers (IHPs), especially by selecting said IPCMs and/or IHPs from a group consisting of sulfonated tetrafluoroethylene copolymers; commercially available Nafion™ and derivatives thereof.

It is another object of the present invention to disclose a method as defined in any of the above, wherein the method further comprising steps of providing the means for deterring biofilm with two or more, either two-dimensional (2D), topologically folded 2D surfaces or three-dimensional (3D) PSSs, each of which of said PSSs consisting of materials containing highly dissociating cationic and/or anionic groups (HDCAs); and, spatially organizing said HDCAs in a manner which minimizes the change of the pH of the TLC's environment, especially a cosmetic article of a foodstuff;

It is another object of the present invention to disclose a method as defined in any of the above, wherein the method further comprising a step of spatially organizing each of said HDCAs in a specific, either 2D or 3D manner, such that the change of the pH of the TLC's environment is minimized.

It is another object of the present invention to disclose a method as defined in any of the above, wherein said step of organizing is provided by a manner selected for a group consisting of (i) interlacing said HDCAs; (ii) overlapping said HDCAs; (iii) conjugating said HDCAs; and (iv) either homogeneously or heterogeneously mixing said HDCAs; and (v) tiling of the same.

It is another object of the present invention to disclose a method as defined in any of the above, wherein the method further comprising a step of disrupting pH homeostasis and/or electrical potential within at least a portion of an LTC by a PSS, while both (i) effectively preserving the pH of said LTC's environment, especially a cosmetic article of a foodstuff; and (ii) minimally affecting the entirety of said LTC's environment; said method is especially provided by minimizing the leaching of either ionized or electrically neutral atoms, molecules or particles (AMP) from the PSS to said LTC's environment.

It is another object of the present invention to disclose a method as defined in any of the above, wherein the method further comprising steps of preferentially disrupting pH homeostasis and/or electrical balance within at least one first confined volume (e.g., target living cells or viruses, TLC), while less disrupting pH homeostasis within at least one second confined volume (e.g., non-target cells or viruses, NTC).

It is another object of the present invention to disclose a method as defined in any of the above, wherein said differentiation between said TLC and NTC is obtained by one or more of the following steps: (i) providing differential ion capacity; (ii) providing differential pH value; (iii) optimizing the PSS to LTC size ratio; and, (iv) designing a differential spatial configuration of said PSS boundaries on top of the PSS bulk; and (v) providing a critical number of PSS' particles (or applicable surface) with a defined capacity per a given volume; and (vi) providing size exclusion means, e.g., mesh, grids etc.

A method for the production of means for deterring biofilm, comprising steps of providing a means for deterring biofilm as defined above; locating the PSS on top or underneath the surface of said means for deterring biofilm; and upon contacting said PSS with a LTC, disrupting the pH homeostasis and/or electrical balance within at least a portion of said LTC while effectively preserving pH & functionality of said surface.

It is another object of the present invention to disclose a method as defined in any of the above, wherein the method further comprising steps of providing the means for deterring biofilm with at least one external proton-permeable surface with a given functionality; providing at least a portion of said surface with at least one PSS, and/or layering at least one PSS on top or underneath said surface; hence killing LTCs or otherwise disrupting vital intracellular processes and/or intercellular interactions of said LTC, while effectively preserving said LTC's environment's pH & functionality.

It is another object of the present invention to disclose a method as defined in any of the above, wherein the method further comprising steps of: providing the means for deterring biofilm with at least one external proton-permeable providing a surface with a given functionality; disposing one or more external proton-permeable layers topically and/or underneath at least a portion of said surface; said one or more layers are at least partially composed of or layered with at least one PSS; and, killing LTCs, or otherwise disrupting vital intracellular processes and/or intercellular interactions of said LTC, while effectively preserving said LTC's environment's pH & functionality.

It is another object of the present invention to disclose a method as defined in any of the above, wherein the method further comprising steps of providing the means for deterring biofilm with at least one PSS; and, providing said PSS with at least one preventive barrier such that a sustained long acting is obtained.

It is another object of the present invention to disclose a method as defined in any of the above, wherein said step of providing said barrier is obtained by utilizing a polymeric preventive barrier adapted to avoid heavy ion diffusion; preferably by providing said polymer as an ionomeric barrier, and particularly by utilizing a commercially available Nafion™ product.

It is hence in the scope of the invention wherein one or more of the following materials are provided: encapsulated strong acidic and strong basic buffers in solid or semi-solid envelopes, solid ion-exchangers (SIEx), ionomers, coated-SIEx, high-cross-linked small-pores SIEx, Filled-pores SIEx, matrix-embedded SIEx, ionomeric particles embedded in matrices, mixture of anionic (acidic) and cationic (basic) SIEx etc.

It is another object of the invention to disclose the PSS as defined in any of the above, wherein the PSS are naturally occurring organic acids compositions containing a variety of carbocsylic and/or sulfonic acid groups of the family, abietic acid (C₂₀H₃₀O₂) such as colophony/rosin, pine resin and alike, acidic and basic terpenes.

It is another object of the present invention to disclose a method for inducing apoptosis in at least a portion of LTCs population in a means for deterring biofilm, especially a means for deterring biofilm of cosmetics and foodstuffs; said method comprising steps of obtaining a means for deterring biofilm as defined above; contacting the PSS with an LTC; and, effectively disrupting the pH homeostasis and/or electrical balance within said LTC such that said LTC's apoptosis is obtained, while efficiently preserving the pH of said LTC's environment and patient's safety.

It is another object of the present invention to disclose a method for avoiding development of LTC's resistance and selecting over resistant mutations, said method comprising steps of obtaining a means for deterring biofilm as defined above; contacting the PSS with an LTC; and, effectively disrupting the pH homeostasis and/or electrical balance within said LTC such that development of LTC's resistance and selecting over resistant mutations is avoided, while efficiently preserving the pH of said LTC's environment and patient's safety.

It is another object of the present invention to disclose a method of regenerating the biocidic properties of a means for deterring biofilm as defined above; comprising at least one step selected from a group consisting of (i) regenerating said PSS; (ii) regenerating its buffering capacity; and (iii) regenerating its proton conductivity.

It is in the scope of the invention wherein the PSS is an insoluble hydrophobic, either anionic, cationic or zwitterionic charged polymer, useful for killing living target cells (LTCs), or otherwise disrupting vital intracellular processes and/or intercellular interactions of the LTC upon contact. It is additionally or alternatively in the scope of the invention, wherein the PSS is an insoluble hydrophilic, anionic, cationic or zwitterionic charged polymer, combined with water-immiscible polymers useful for killing living target cells (LTCs), or otherwise disrupting vital intracellular processes and/or intercellular interactions of the LTC upon contact. It is further in the scope of the invention, wherein the PSS is an insoluble hydrophilic, either anionic, cationic or zwitterionic charged polymer, combined with water-immiscible either anionic, cationic of zwitterionic charged polymer useful for killing living target cells (LTCs), or otherwise disrupting vital intracellular processes and/or intercellular interactions of the LTC upon contact.

It is also in the scope of the invention wherein the PSS is adapted in a non-limiting manner, to contact the living target cell either in a bulk or in a surface; e.g., at the outermost boundaries of an organism or inanimate object that are capable of being contacted by the PSS of the present invention; at the inner membranes and surfaces of animals and plants, capable of being contacted by the PSS by any of a number of transdermal delivery routes etc; at the bulk, either a bulk provisioned with stirring or nor etc.

It is further in the scope of the invention wherein either (i) a PSS or (ii) an article of manufacture comprising the PSS also comprises an effective measure of at least one additive.

### BRIEF DESCRIPTION OF THE DRAWINGS

In order to understand the invention and to see how it may be implemented in practice, a plurality of preferred embodiments will now be described, by way of non-limiting example only, with reference to the accompanying drawing, in which

FIG. 1 illustrates a bacterial test taken from partially coated glass slide after the first cycle of incubation/evaporation with E. coli. Wet swab sampled were taken from coated and uncoated slides, spread on a TSA plate, and incubated for 24 h;

FIG. 2 illustrates bacterial counts of coated and uncoated glass slide after first cycle of incubation/evaporation with E. coli. Bacterial samples were taken using cotton swab. Following swabbing, the samples were vortexed vigorously in 500 μl PBS diluted by tenfold-dilutions, inoculated on TSA plates (100 μl), incubated (24 h, 30° C.) and counted;

FIG. 3 illustrates a partially coated glass slide after the first cycle of incubation with E. coli;

FIG. 4 illustrates a representative image of partially coated glass slide after 4 cycles of incubation/evaporation with cell suspension of E. coli. Microscope glass slide was coated with Nafion™ active material (0.05 gr/cm² Nafion™ solution in 20% aliphatic alcohol in 4% polyacryl amid gel (PAAG)), and placed in Petri dish with inoculated with E. coli inoculums (25 ml of E. coli inoculum in LB, approximately 1×10⁷/ml), covered with plastic lid, and incubated at 30° C.;

FIG. 5 illustrates a bacterial counts of coated and uncoated glass slide after 4 cycles of incubation/evaporation with E. coli. Bacterial samples were taken using cotton swab. Following swabbing, the samples were vortexed vigorously in 500 μl PBS diluted by tenfold-dilutions, inoculated on TSA plates (100 μl), incubated (24 h, 30° C.) and counted; and

FIG. 6 illustrates a bacterial counts of glass slides coated and uncoated with 0.01 gr/cm² sulfonated silica in 4% PAAG (Sigma-Aldrich, 57221-U, Discovery® DSC-SCX SPE Bulk Packing), a self-made analog of polymerically bonded, benzene sulfonic acid on silica nanoparticles. After the incubation with E. coli Bacterial samples were taken using cotton swab. Following swabbing, the samples were vortexed vigorously in 500 μl PBS diluted by tenfold-dilutions, inoculated on TSA plates (100 μl), incubated (24 h, 30° C.) and counted.

### DESCRIPTION OF THE PREFERRED EMBODIMENTS

The following specification taken in conjunction with the drawings sets forth the preferred embodiments of the present invention. The embodiments of the invention disclosed herein are the best modes contemplated by the inventors for carrying out their invention in a commercial environment, although it should be understood that various modifications can be accomplished within the parameters of the present invention.

The term ‘contact’ refers hereinafter to any direct or indirect contact of a PSS with a confined volume (living target cell or virus—LTC), wherein the PSS and LTC are located adjacently, e.g., wherein the PSS approaches either the internal or external portions of the LTC; further wherein the PSS and the LTC are within a proximity which enables (i) an effective disruption of the pH homeostasis and/or electrical balance, or (ii) otherwise disrupting vital intracellular processes and/or intercellular interactions of the LTC.

The terms ‘effectively’ and ‘efficiently’ refer hereinafter to an effectiveness of over 10%, additionally or alternatively, the term refers to an effectiveness of over 50%; additionally or alternatively, the term refers to an effectiveness of over 80%. It is in the scope of the invention, wherein for purposes of killing LTCs, the term refers to killing of more than 50% of the LTC population in a predetermined time, e.g., 10 min.

The term ‘additives’ refers hereinafter to one or more members of a group consisting of biocides e.g., organic biocides such as tea tree oil, rosin, abietic acid, terpens, rosemary oil etc, and inorganic biocides, such as zinc oxides, cupper and mercury, silver salts etc, markers, biomarkers, dyes, pigments, radio-labeled materials, glues, adhesives, lubricants, medicaments, sustained release drugs, nutrients, peptides, amino acids, polysaccharides, enzymes, hormones, chelators, multivalent ions, emulsifying or de-emulsifying agents, binders, fillers, thickfiers, factors, co-factors, enzymatic-inhibitors, organoleptic agents, carrying means, such as liposomes, multilayered vesicles or other vesicles, magnetic or paramagnetic materials, ferromagnetic and non-ferromagnetic materials, biocompatibility-enhancing materials and/or biodegradating materials, such as polylactic acids and polyglutaminc acids, anticorrosive pigments, anti-fouling pigments, UV absorbers, UV enhancers, blood coagulators, inhibitors of blood coagulation, e.g., heparin and the like, or any combination thereof.

The term ‘particulate matter’ refers hereinafter to one or more members of a group consisting of nano-powders, micrometer-scale powders, fine powders, free-flowing powders, dusts, aggregates, particles having an average diameter ranging from about 1 nm to about 1000 nm, or from about 1 mm to about 25 mm.

The term about’ refers hereinafter to ±20% of the defined measure.

The present invention relates to materials, compositions and methods for biofilm prevention and treatment in water systems (e.g. water storage, water treatment and water supply and transport systems) based on preferential proton and/or hydroxyl-exchange between the cell and strong acids and/or strong basic materials and compositions. The materials and compositions of the present invention exert their antimicrobial and anti-biofilm effect via a titration-like process in which the said cell is coming into contact with strong acids and/or strong basic buffers and the like: encapsulated strong acidic and strong basic buffers in solid or semi-solid envelopes, solid ion-exchangers (SIEx), ionomers, coated-SIEx, high-cross-linked small-pores SIEx, Filled-pores SIEx, matrix-embedded SIEx, Ionomeric particles embedded in matrices, mixture of anionic (acidic) and cationic (basic) SIEx etc. This process leads to disruption of the cell pH-homeostasis and consequently to cell death. The proton conductivity property, the volume buffer capacity and the bulk activity are pivotal and crucial to the present invention. The presence or incorporation of barriers that can selectively allow transport of protons and hydroxyls but not of other competing ions to and/or from the SIEx surface eliminates or substantially reduces the ion-exchange saturation by counter-ions, resulting in sustained and long acting cell killing activity of the materials and compositions of the current invention.

The materials and compositions of the current invention include but not limited to all materials and compositions disclosed in PCT application No. PCT/IL2006/001262. The above mentioned materials and compositions of PCT/IL2006/001262 modified in such a way that these said compositions are ion-selective by, for example: coating them with a selective coating, or ion-selective membrane; coating or embedding in high-cross-linked size excluding polymers etc. Strong acidic and strong basic buffers encapsulated in solid or semi-solid envelopes. SIEx particles-coated and non-coated, alone or in a mixture, embedded in matrices so as to create a pH-modulated polymer. SIEx particles-coated and non-coated, embedded in porous ceramic or glass water permeable matrices. Polymers which are alternately tiled with areas of high and low pH to create a mosaic-like polymer with an extended cell-killing spectrum. In addition to ionomers disclosed in the above mentioned PCT No. PCT/IL2006/001262, other ionomers can be used in the current invention as cell-killing materials and compositions. These may include, but certainly not limited to, for example: sulfonated silica, sulfonated polythion-ether sulfone (SPTES), sulfonated styrene-ethylene-butylene-styrene (S-SEBS), polyether-ether-ketone (PEEK), poly (arylene-ether-sulfone) (PSU), Polyvinylidene Fluoride (PVDF)-grafted styrene, polybenzimidazole (PBI) and polyphosphazene, proton-exchange membrane made by casting a polystyrene sulfonate (PSS) solution with suspended micron-sized particles of cross-linked PSS ion exchange resin.

It is in the scope of the invention, wherein the means for water treatment comprises an insoluble PSS in the form of a polymer, ceramic, gel, resin or metal oxide is disclosed. The PSS is carrying strongly acidic or strongly basic functional groups (or both) adjusted to a pH of about <4.5 or about >8.0. It is in the scope of the invention, wherein the insoluble PSS is a solid buffer.

It is also in the scope of the invention wherein material's composition is provided such that the groups are accessible to water whether they are on the surface or in the interior of the PSS. Contacting a living cell (e.g., bacteria, fungi, animal or plant cell) with the PSS kills the cell in a time period and with an effectiveness depending on the pH of the PSS, the mass of PSS contacting the cell, the specific functional group(s) carried by the PSS, and the cell type. The cell is killed by a titration process where the PSS causes a pH change within the cell. The cell is often effectively killed before membrane disruption or cell lysis occurs. The PSS kills cells without directly contacting the cells if contact is made through a coating or membrane which is permeable to water, H+ and OH− ions, but not other ions or molecules. Such a coating also serves to prevent changing the pH of the PSS or of the solution surrounding the target cell by diffusion of counterions to the PSS's functional groups. It is acknowledged in thos respect that prior art discloses cell killing by strongly cationic (basic) molecules or polymers where killing probably occurs by membrane disruption and requires contact with the strongly cationic material or insertion of at least part of the material into the outer cell membrane.

It is also in the scope of the invention wherein an insoluble polymer, ceramic, gel, resin or metal oxide carrying strongly acid (e.g. sulfonic acid or phosphoric acid) or strongly basic (e.g. quaternary or tertiary amines) functional groups (or both) of a pH of about <4.5 or about >8.0 is disclosed. The functional groups throughout the PSS are accessible to water, with a volumetric buffering capacity of about 20 to about 100 mM H⁺/l/pH unit, which gives a neutral pH when placed in unbuffered water (e.g., about 5<pH>about 7.5) but which kills living cells upon contact.

It is also in the scope of the invention wherein the insoluble polymer, ceramic, gel, resin or metal oxide as defined above is coated with a barrier layer permeable to water, H⁺ and OH⁻ ions, but not to larger ions or molecules, which kills living cells upon contact with the barrier layer.

It is also in the scope of the invention wherein the insoluble polymer, ceramic, gel, resin or metal oxide as defined above is provided useful for killing living cells by inducing a pH change in the cells upon contact.

It is also in the scope of the invention wherein the insoluble polymer, ceramic, gel, resin or metal oxide as defined above is provided useful for killing living cells without necessarily inserting any of its structure into or binding to the cell membrane.

It is also in the scope of the invention wherein the insoluble polymer, ceramic, gel, resin or metal oxide as defined above is provided useful for killing living cells without necessarily prior disruption of the cell membrane and lysis.

It is also in the scope of the invention wherein the insoluble polymer, ceramic, gel, resin or metal oxide as defined above is provided useful for causing a change of about <0.2 pH units of a physiological solution or body fluid surrounding a living cell while killing the living cell upon contact.

It is also in the scope of the invention wherein the insoluble polymer, ceramic, gel, resin or metal oxide as defined above is provided in the form of shapes, a coating, a film, sheets, beads, particles, microparticles or nanoparticles, fibers, threads, powders and a suspension of these particles.

All of the above mentioned materials and compositions of the current invention can be cast, molded or extruded and be used as particles in suspension, spray, as membranes, coated films, fibers or hollow fibers, particles linked to or absorbed on fibers or hollow fibers, incorporated in filters or tubes and pipes etc.

### Experiment 1

Static biofilm

### Materials and Methods

The antibacterial properties of Nafion™ were tested using static biofilm experiment. Microscopic glass slides were coated with Nafion™ active material (0.01 gr/cm² Nafion™ solution in 20% aliphatic alcohol in 4% polyacryl amid gel (PAAG)). The glass slides were placed in culture dish with E. coli inoculums, 25 ml E. coli inoculum in LB (E. coli, approximately 1×107/ml), uncovered, 30° C. without shaking. The culture medium was evaporated and followed by a bacterial load evaluation using seeding of samples obtained from the coated and uncoated slides.

### Results

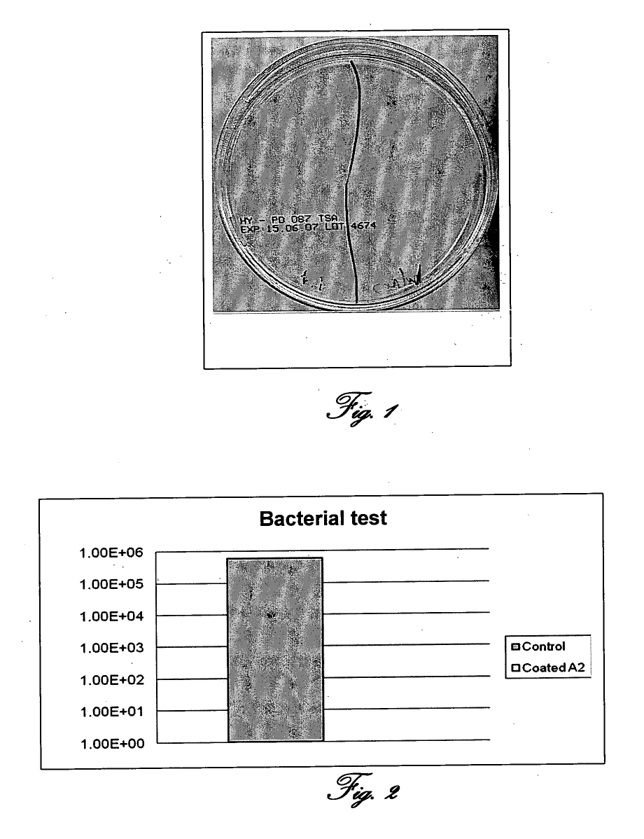

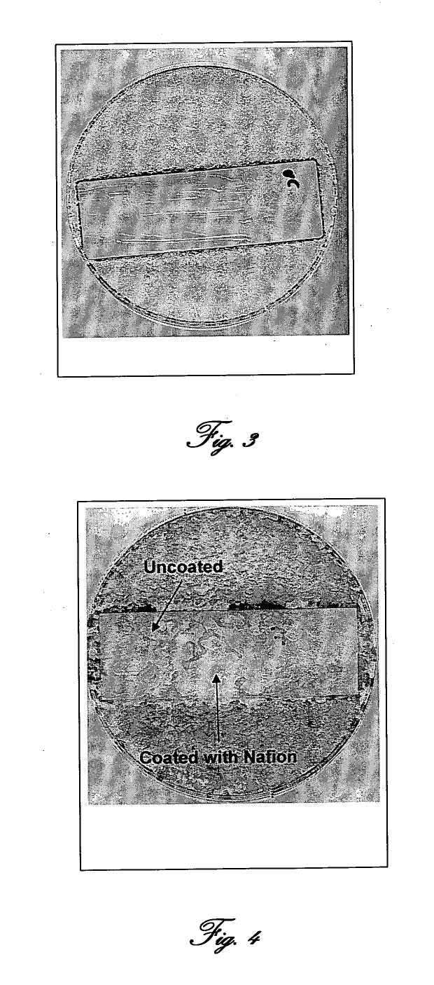

Reference is now made to FIG. 1, presenting bacterial test taken from partially coated glass slide after the first cycle of incubation/evaporation with E. coli; to FIG. 2, illustrating bacterial counts of coated and uncoated glass slide after incubation/evaporation with E. coli; and to FIG. 3, disclosing partially coated glass slide after the first cycle of incubation with E. coli.

A bacterial sample was taken from a coated and uncoated glass seeded on agar plate and incubated (30° C.). The sample obtained from the uncoated glass developed into substantial bacterial colonies (>5×10⁵ cfu/ml) while the sample obtained from the coated areas did not show any bacterial sign (FIGS. 1 & 2). A representative picture of the partially coated slide can be seen in FIG. 3.

### Experiment 2

Static biofilm

### Materials and Methods

The antibacterial properties of Nafion™ were tested using static biofilm experiment. Microscopic glass slides were coated with Nafion™ active material (0.05 gr/cm² Nafion™ solution in 20% aliphatic alcohol in 4% polyacryl amid gel (PAAG)), and placed in culture dish with E. coli inoculums, 25 ml E.coli inoculum in LB (E. coli, approximately 1×10⁷/ml), covered with plastic lid, 30° C. without shaking. The culture medium was evaporated and followed by a bacterial load evaluation using seeding of samples obtained from the coated and uncoated slides. The procedure was repeated for 4 cycles (21 days).

### Results

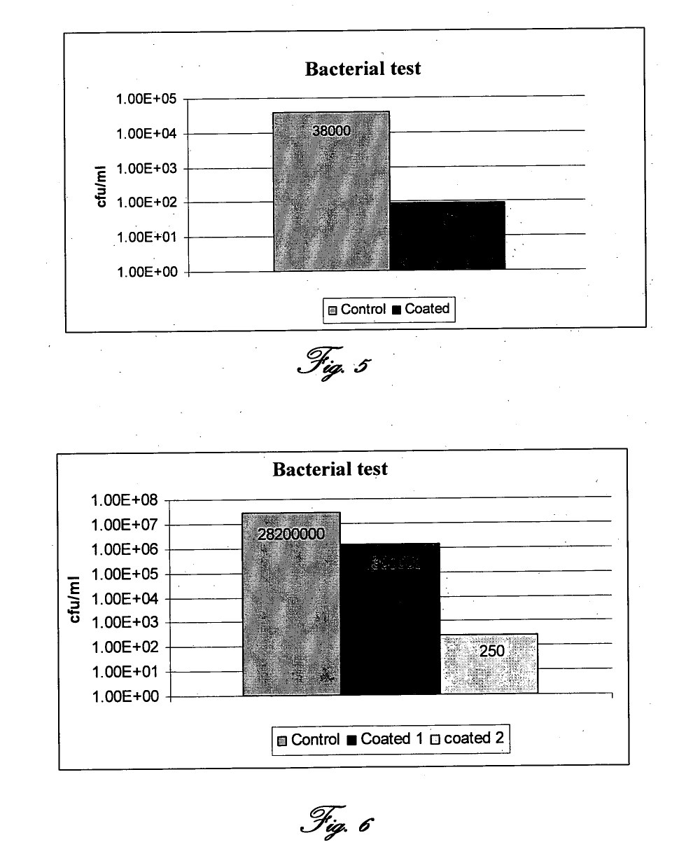

Reference is now made to FIG. 4, presenting representative image of partially coated glass slide after 4 cycles of incubation/evaporation with E. coli; and to FIG. 5, showing bacterial counts of coated and uncoated glass slide after 4 cycles of incubation/evaporation with E. coli.

After the 4th cycle a bacterial sample was taken from a coated and uncoated glass seeded on agar plate and incubated (30° C.). The sample obtained from the uncoated glass developed into substantial bacterial colonies (3.8×10⁴ cfu/ml) while the sample obtained from the coated areas showed reduction in the bacterial load (90 cfu/ml) (FIG. 5). A representative picture of a partially coated slide can be seen in FIG. 4.

### Experiment 3

Static biofilm

### Materials and Methods

The antibacterial properties of sulfonated silica were tested using static biofilm experiment. Microscopic glass slides were coated with 0.01 gr/cm² sulfonated silica in 4% PAAG. (Sigma-Aldrich, 57221-U, Discovery® DSC-SCX SPE Bulk Packing), a self-made analog of polymerically bonded, benzene sulfonic acid on silica nano-particles, cf. http://www.sigmaaldrich.com/catalog/search/ProductDetail/SUPELCO/57221-U); prepared by patent EP0386926; U.S. Pat. No. 4,933,372; or coated with Potassium Dodecylsulfate 0.05 gr/cm². Coated slides and uncoated control slides were placed in a culture dish and covered with 25 ml (E. coli, 40×10⁹/ml, 25° C.). The culture medium was evaporated and followed by a bacterial load evaluation using seeding of samples obtained from the coated and uncoated slides.

### Results

Reference is now made to FIG. 6, presenting bacterial counts of coated and uncoated glass slide after first cycle of incubation/evaporation with E. coli.

After the first cycle a bacterial sample was taken from a sulfonated silica-coated glass, the Potassium Dodecylsulfate-coated glass and from an uncoated control glass seeded on agar plate and incubated (30° C.). The sample obtained from the uncoated glass developed into substantial bacterial colonies (2.8×10⁷ cfu/ml). A minor and a large bacteriological reduction were observed from sample obtained from the sulfonated silica-coated glass (1.3×10⁶ cfu/ml) and from the Potassium Dodecylsulfate -coated glass ((250 cfu/ml)) respectively (FIG. 6).

1-35. (canceled)

36. A means for deterring biofilm in water supply systems, said means for deterring biofilm adapted for killing cells contained within a given volume, said means for deterring biofilm comprising at least one charged polymer, said at least one charged polymer characterized, when in contact with a water-containing environment, as:
a. carrying strongly acid and/or strongly basic functional groups;
b. having a pH of less than about 4.5 or greater than about 8.0;
c. capable of generating an electrical potential within the confined volume of said cell sufficient to disrupt effectively the pH homeostasis and/or electrical balance within said confined volume of said cell; and,
d. being in a form chosen from the group consisting of (i) H⁺ and (ii) OH⁻;
wherein said charged polymer is adapted to preserve the pH of said cell's environment.

37. The means for deterring biofilm of claim 36, further characterized, when in contact with a water-containing environment, as having a buffering capacity of about 20 to about 100 mM H⁺/L/pH unit.

38. The means for deterring biofilm of claim 36, further characterized, when in contact with a water-containing environment, by at least one characteristic chosen from the group consisting of (a) sufficiently water-insoluble such that at least 99.9% remains undissolved at equilibrium; (b) sufficiently resistant to leaching such that the total concentration of material leached from said composition of matter into said water-containing environment does not exceed 1 ppm; (c) sufficiently inert such that at least one parameter of said water-containing environment chosen from the group consisting of (i) concentration of at least one predetermined water-soluble substance; (ii) particle size distribution; (iii) rheology; (iv) toxicity; (v) color; (vi) taste; (vii) smell; and (viii) texture remains unaffected according to preset conditions, said conditions adapted for and appropriate to said particular environment.

39. The means for deterring biofilm of claim 36, further characterized, when in contact with said water-containing environment, as being sufficiently inert such that the toxicity in said water-containing environment as defined by at least one parameter chosen from the group consisting of (a) LD₅₀ and (b) ICT₅₀ remains unaffected according to preset conditions, said conditions adapted for and appropriate to said particular environment.

40. The means for deterring biofilm of claim 36, further comprising at least one polymer chosen from the group consisting of (a) polyvinyl alcohol; (b) polystyrene sulfonate; and (c) polypropylene polystyrene-divinylbenzene.

41. The means for deterring biofilm of claim 40, wherein at said at least one polymer contains at least one functional group chosen from the group consisting of SO₃H and H₂N(CH₃).

42. The means for deterring biofilm of claim 36, further comprising hydrophilic additives chosen from the group consisting of proton conductive materials (PCMs) and hydrophilic polymers (HPs); further wherein said PCMs and HPs are chosen from the group consisting of (a) sulfonated tetrafluoroethylene copolymers; (b) sulfonated materials chosen from the group consisting of silica, polythion-ether sulfone (SPTES), styrene-ethylene-butylene-styrene (S-SEBS), polyether-ether-ketone (PEEK), poly(arylene-ether-sulfone) (PSU), polyvinylidene fluoride (PVDF)-grafted styrene, polybenzimidazole (PBI), and polyphosphazene; and (c) proton-exchange membranes made by casting a polystyrene sulfonate (PSSnate) solution with suspended micron-sized particles of cross-linked PSSnate ion exchange resin.

43. The means for deterring biofilm of claim 36, comprising two or more charged polymers chosen from the group consisting of two-dimensional charged polymers and three-dimensional (3D) charged polymers, each of which of said charged polymers comprises materials containing cationic and/or anionic groups capable of dissociation and spatially organized in a manner adapted to preserve the pH of said water-containing environment according to preset conditions; said spatial organization chosen from the group consisting of (a) interlacing; (b) overlapping; (c) conjugating; (d) homogeneously mixing; (e) heterogeneously mixing; and (f) tiling.

44. The means for deterring biofilm of claim 36, further comprising a surface with a given functionality and at least one external proton-permeable layer, each of which of said at least one external proton-permeable layers is disposed on at least a portion of said surface.

45. The means for deterring biofilm of claim 36, comprising at least one charged polymer and at least one barrier adapted to prevent heavy ion diffusion.

46. The means for deterring biofilm of claim 36, wherein said means for deterring biofilm is in the form of an insert of dimensions adapted to allow mounting within an article of manufacture of predetermined dimensions, said mounting chosen from the group consisting of reversible mounting and permanent accommodation.

47. The means for deterring biofilm of claim 36 designed as a continuous barrier, said barrier selected from the group consisting of 2D or 3D membranes, filters, meshes, nets, sheet-like members, or a combination thereof.

48. The means for deterring biofilm of claim 36, wherein said means for deterring biofilm is in a form chosen from the group consisting of (a) powder; (b) gel; (c) suspension; (d) spray; (e) resin; (f) coating; (g) film; (h) sheet; (i) bead; (j) particle; (k) microparticle; (I) nanoparticle; (m) fiber; (n) thread; (o) mesh.

49. The means for deterring biofilm of claim 36, wherein said means for deterring biofilm is incorporated into a filter.

50. The means for deterring biofilm of claim 36, wherein said means for deterring biofilm is incorporated into a tube and/or pipe.

51. The means for deterring biofilm of claim 36, further characterized by at least one of the following:
a. capacity for absorbing or releasing protons capable of regeneration;
b. buffering capacity capable of regeneration;
c. proton conductivity capable of regeneration.

52. A method for increasing the rate of death of living cells and/or decreasing the rate of reproduction of living cells within a water containing-environment, comprising the steps of:
a. providing a means for deterring biofilm comprising at least one charged polymer, said at least one charged polymer characterized, when in contact with said water-containing environment, as:
i. carrying strongly acid and/or strongly basic functional groups;
ii. having a pH of less than about 4.5 or greater than about 8.0;
iii. capable of generating an electrical potential within the confined volume of said cell sufficient to disrupt effectively the pH homeostasis and/or electrical balance within said confined volume of said cell; and,
iv. being in a form chosen from the group consisting of (i) H⁺ and (ii) OH⁻; and,
b. placing said means for deterring biofilm in contact with said water-containing environment.

53. The method of claim 52, wherein said step (a) further comprises the step of providing said charged polymer with predetermined water permeability, proton conductivity, and/or wetting characteristics, and further wherein said water permeability, proton conductivity, and/or wetting characteristics are provided by at least one substance selected from the group consisting of proton conductive materials (PCMs) and hydrophilic polymers (HPs).

54. The method of claim 53, wherein said step of providing said charged polymer with predetermined water permeability, proton conductivity, and/or wetting characteristics, and further wherein said water permeability, proton conductivity, and/or wetting characteristics are provided by at least one substance selected from the group consisting of proton conductive materials (PCMs) and hydrophilic polymers (HPs) further comprises a step of choosing said PCMs and HPs from the group consisting of (a) sulfonated tetrafluoroethylene copolymers; (b) sulfonated materials chosen from the group consisting of silica, polythion-ether sulfone (SPTES), styrene-ethylene-butylene-styrene (S-SEBS), polyether-ether-ketone (PEEK), poly(arylene-ether-sulfone) (PSU), polyvinylidene fluoride (PVDF)-grafted styrene, polybenzimidazole (PBI), and polyphosphazene; (c) proton-exchange membranes made by casting a polystyrene sulfonate (PSSnate) solution with suspended micron-sized particles of cross-linked PSSnate ion exchange resin; and derivatives thereof.

55. The method of claim 52, further comprising a step of providing at least one polymer chosen from the group consisting of (a) polyvinyl alcohol; (b) polystyrene sulfonate; and (c) polypropylene polystyrene-divinylbenzene.

56. The method of claim 52, wherein said step of providing at least one polymer further comprises a step of providing at least one polymer that contains at least one functional group chosen from the group consisting of SO₃H and H₂N(CH₃).

57. The method of claim 52, further comprising a step of providing two or more charged polymers chosen from the group consisting of two-dimensional charged polymers and three-dimensional (3D) charged polymers, each of which of said charged polymers comprises materials containing cationic and/or anionic groups capable of dissociation and spatially organized in a manner adapted to preserve the pH of said water-containing environment according to preset conditions; said spatial organization chosen from the group consisting of (a) interlacing; (b) overlapping; (c) conjugating; (d) homogeneously mixing; (e) heterogeneously mixing; and (f) tiling.

58. The method of claim 52, further comprising a step of spatially organizing each of said functional groups in a manner selected from (a) interlacing; (b) overlapping; (c) conjugating; (d) homogeneously mixing; (e) heterogeneously mixing; and (f) any combination of the above.

59. The method of claim 52, further comprising an additional step of providing said charged polymer with an ionomeric barrier layer comprising a sulfonated tetrafluoroethylene copolymer, said barrier adapted to avoid heavy ion diffusion.

60. A method of production of a means for deterring biofilm, comprising the steps of:
a. providing at least one charged polymer, said at least one charged polymer characterized, when in contact with said water-containing environment, as:
i. carrying strongly acid and/or strongly basic functional groups;
ii. having a pH of less than about 4.5 or greater than about 8.0;
iii. capable of generating an electrical potential within the confined volume of said cell sufficient to disrupt effectively the pH homeostasis and/or electrical balance within said confined volume of said cell; and,
iv. being in a form chosen from the group consisting of (i) H⁺ and (ii) OH⁻; and,
b. adapting said charged polymer to a form chosen from the group consisting of (a) powder; (b) gel; (c) suspension; (d) spray; (e) resin; (f) coating; (g) film; (h) sheet; (i) bead; (j) particle; (k) microparticle; (l) nanoparticle; (m) fiber; (n) thread; (o) shape; (p) membrane; (q) coated film; (r) hollow fiber; (s) particle linked to a fiber; and (t) particle adsorbed on a fiber.

61. The method of claim 60, wherein said step of providing at least one electrolyte charged polymer characterized, when in contact with said water-containing environment, by at least one characteristic chosen from the group consisting of (a) sufficiently water-insoluble such that at least 99.9% remains undissolved at equilibrium; (b) sufficiently resistant to leaching such that the total concentration of material leached from said composition of matter into said water-containing environment does not exceed 1 ppm; (c) sufficiently inert such that at least one parameter of said water-containing environment chosen from the group consisting of (i) concentration of at least one predetermined water-soluble substance; (ii) particle size distribution; (iii) rheology; (iv) toxicity; (v) color; (vi) taste; (vii) smell; and (viii) texture remains unaffected according to preset conditions, said conditions adapted for and appropriate to said particular environment.

62. The method of claim 60, wherein said step of providing at least one electrolyte further comprises the step of providing a charged polymer characterized, when in contact with said water-containing environment, as being sufficiently inert such that the toxicity said water-containing environment as defined by at least one parameter chosen from the group consisting of (a) LD₅₀ and (b) ICT₅₀ remains unaffected according to preset conditions, said conditions adapted for and appropriate to said particular environment.

63. The method of claim 60, further comprising steps of:
c. providing at least one external proton-permeable surface with a predetermined functionality; and
d. layering at least a portion of said proton-permeable surface with at least one of said charged polymer.

64. The method of claim 60, wherein said step of providing at least one polymer further comprises a step of providing at least one polymer chosen from the group consisting of (a) polyvinyl alcohol; (b) polystyrene sulfonate; and (c) polypropylene polystyrene-divinylbenzene.

65. The method of claim 60, wherein said step of providing at least one polymer that contains at least one functional group chosen from the group consisting of SO₃H and H₂N(CH₃).

66. A method for regenerating the biocidic properties of a means for deterring biofilm as defined in claim 36, said method comprising at least one step chosen from the group consisting of (a) regenerating said means for deterring biofilm's proton absorbing and/or releasing capacity; (b) regenerating said means for deterring biofilm's buffering capacity; and (c) regenerating the proton conductivity of said means for deterring biofilm.
